(19) United States
(12) Patent Application Publication
(10) Pub. No.: US 2023/0300508 A1
(43) Pub. Date: Sep. 21, 2023

(54) WIRELESS EARPHONE

(71) Applicant: Olive Union, Inc., Tokyo (JP)

(72) Inventor: Myung Geun Song, Tokyo (JP)

(73) Assignee: Olive Union, Inc., Tokyo (JP)

(21) Appl. No.: 18/007,971

(22) PCT Filed: Jun. 4, 2020
(86) PCT No.: PCT/JP2020/022128

§ 371 Date: May 18, 2023

(51) Int. Cl. H04R 1/10

(52) U.S. Cl. CPC H04R 1/1016; A61N 1/36038; H04R 2420/07

(57) ABSTRACT

To provide wireless earphones that are capable of reducing tinnitus by noninvasively applying electrical stimulation to brain cells regardless of the condition of auditory cells. Wireless earphones 100 that have a left-right pair of a left earphone 100L and a right earphone 100R which are directly or indirectly connected to a server 300 via a network NW, obtain audio data from the server 300, and output the same, the wireless earphones 100 comprising: a first electrode 100a that is provided on a predetermined position on the left earphone 100L, and is capable of contacting a vicinity of the left temporal region of a user wearing the left earphone 100L; a second electrode 100b that is provided on a predetermined position on the right earphone 100R, and is capable of contacting a vicinity of the right temporal region of the user wearing the right earphone 100R; and an electrical stimulation unit that applies a predetermined voltage between the first electrode 100a and the second electrode 100b, and electrically stimulates the brain cells of the user.

### TECHNICAL FIELD

The present disclosure relates to wireless earphones.

Conventionally, devices and methods for the treatment of tinnitus are known.

### BACKGROUND ART

For example, Patent Document 1 discloses a method for generating a therapeutic sound for treating tinnitus symptoms.

Japanese Patent Publication 2016-518151

However, since the method disclosed in Patent Document 1 attempts to reduce tinnitus by the pronunciation of the therapeutic sound, strict hearing is required. For this reason, for example, it is not suitable when used by a person who is damaging auditory cells.

### DETAILED DESCRIPTION OF THE INVENTION

### Techinical Problem

Therefore, the purpose of this disclosure is to provide wireless earphones that can reduce tinnitus by noninvasively electrical stimulation to brain cells, no matter what the auditory cell is.

### Technical Solution

In one aspect of the present disclosure, a wireless earphone having a left and right pair of left and right earphones that are directly or indirectly connected to the server via the network and acquire and output sound data from the server, provided in a predetermined position of the left earphone, a first electrode that can come into contact with the left head of the user wearing the left earphone; Provided in a predetermined position of the right earphone, a second electrode that can come into contact with the vicinity of the right head of the user wearing the right earphone and a predetermined voltage is applied between the first electrode and the second electrode, and an electrical stimulation unit that electrically stimulates the user’s brain cells.

### Advantageous Effects of the Invention

According to the present disclosure, no matter what the auditory cell is, it is possible to provide wireless earphones that can noninvasively electrically stimulate brain cells and reduce tinnitus.

### DESCRIPTION OF THE DRAWINGS

FIG. 1 is a block configuration diagram showing the first embodiment of the present disclosure.

FIG. 2 is a functional block configuration diagram of the wireless earphone 100 of FIG. 1.

FIG. 3 is a functional block configuration diagram showing the user terminal 200.

FIG. 4 is a functional block configuration diagram of the server 300.

FIG. 5 shows the process flow for adjusting the wireless earphones executed by the system.

FIG. 6 shows sound and / or current stimulation after applying a series of tinnitus reduction methods.

FIG. 7 shows a sound similar to tinnitus is selected from the table of intensity (db) and frequency (Hz).

### BEST MODE

Hereinafter, embodiments of the present disclosure will be described with reference to drawings. Note that the embodiment described below does not unreasonably limit the content of the present disclosure described in the scope of claim, and not all of the components shown in the embodiment are essential components of the present disclosure. Alternatively, in the accompanying drawing, the same or similar elements are accompanied by the same or similar reference codes and names, and overlapping descriptions of the same or similar elements may be omitted in the description of each embodiment. Furthermore, the features shown in each embodiment can also be applied to other embodiments as long as they do not contradict each other.

### The First Embodiment

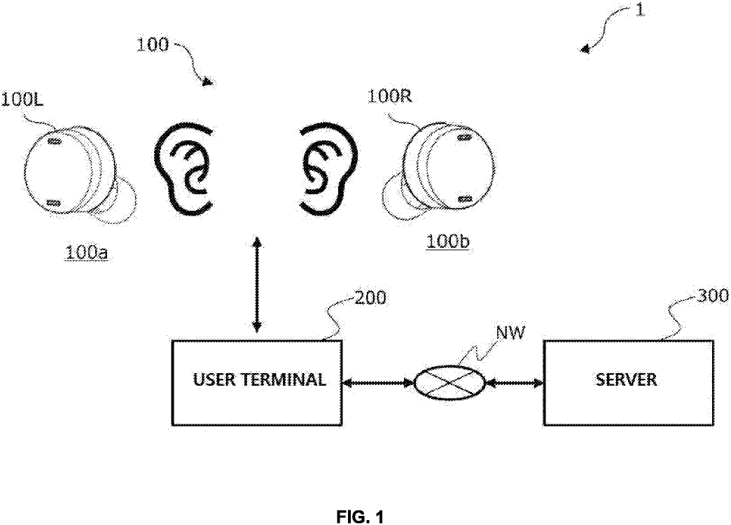

FIG. 1 is a block configuration diagram showing the first embodiment of the present disclosure. In the first embodiment, for example, a wireless earphone 100 used by the user, a user terminal 200 owned by the user, and a server 300 in which the user terminal 200 is connected via the network NW. The network NW is composed of the Internet, an intranet, a wireless LAN (Local Area Network) or a WAN (Wide Area Network), and the like.

In FIG. 1, sign 1 shows a transcranial current stimulation system according to the first embodiment of the present disclosure, detects the EEG voltage from the user’s scalp, and at the same time extracts a weak stimulation current according to the EEG voltage from the head or outputs it to the head via the scalp. In practice, the transcranial current stimulation system 1 includes two electrodes 100a and 100b that can be affixed to a predetermined position of the user’s scalp, and resistance R (not shown) as an irritating current identification means connected between the electrodes 100a and 100b.

Here, it can be considered that an electroencephalogram signal source (for example, internal impedance Z) exists in the head, for example, when two electrodes 100a and 100b are attached to the scalp and the resistance value is between these electrodes 100a and 100b, it can be seen that the current of V / (Z + R) flows into the resistance from the theorem of Feng AndeBnan. Here, V is the voltage when the electrodes 100a and 100b are in an open state. The transcranial current stimulation system 1 of the present disclosure uses the current obtained in conjunction with the electroencephalo frequency as an excitation current using such a principle.

For example, the wireless earphone 100 performs a decrease or decrease in volume or noise cancellation, adjustment of gain (amplification amount), and executes various functions mounted on the input sound. The wireless earphone 100 has a pair of left and right left earphones 100L and a right earphone 100R that acquires and outputs sound data from the server 300. In the predetermined position of the left earphone 100L, a first electrode 100a (anode) that can come into contact with the user’s left head periphery of the user wearing the left earphone 100L is provided. In the predetermined position of the right earphone 100R, a second electrode 100b (cathode) that can come into contact with the right head of the user wearing the right earphone 100R is provided.

The user terminal 200 is a user-owned terminal, for example, an information processing device such as a personal computer or a tablet terminal, but may be configured with a smartphone, a mobile phone, a PDA, or the like.

The server 300 is a device that transmits and receives information to the user terminal 200 via a network NW and computes the received information, for example, a general-purpose computer such as a workstation or personal computer, or may be logically realized by cloud computing. In the present embodiment, one is exemplated as a server device for convenience of explanation, but may be a plurality of units, not limited thereof.

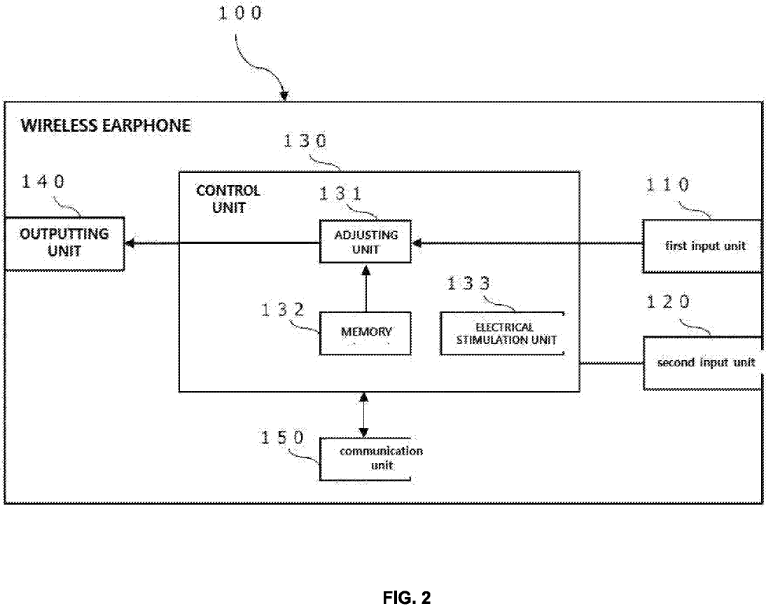

FIG. 2 is a functional block configuration diagram of the wireless earphone 100 of FIG. The wireless earphone 100 includes a first input unit 110, a second input unit 120, a control unit 130, and an output unit 140 and a communication unit 150. The control unit 130 comprises an adjustment unit 131, a storage unit 132, and an electrical stimulation unit 133. Further, although not shown, various sensors such as touch sensors may be provided, and the wireless earphone 100 may be operated by directly tapping or the like.

The first input unit 110 and the second input unit 120 are, for example, a microphone and an A / D converter (not shown). The first input unit 110 is disposed, for example, on the side close to the user’s mouth, in particular acquires audio including the user’s voice and converts it into a digital signal, and the second input unit 120 is disposed on a side far from the user’s mouth, for example, in particular, the surrounding sound including the surrounding ambient sound is acquired and converted into a digital signal. In the first embodiment, it was a configuration having two input portions, but is not limited there to, for example, one may be one, or may be three or more plurality.

The control unit 130 controls the overall operation of the wireless earphone 100, and is composed of, for example, a CPU (Central Processing Unit). The adjustment unit 131 is, for example, a DSP (Digital Sound Processor), and the DSP is adjusted by a set of parameters (a group of variables for generating sound) stored in the storage unit 132, for example, so that the sound received from the first input unit is easier to hear, and more specifically, a plurality of predetermined frequencies (eg, The gain (amplification amount) is adjusted for every 8 channels or 16 channels).

The storage unit 132 may store a set of parameters set by a test such as initial setting, or a parameter set based on the analysis results described later may be stored. These parameter sets may be used alone for adjustment by the adjustment unit 131 or may be used in a composite manner. For example, the storage unit 132 may store profile information about the user’s tinnitus. The profile information can be, for example, any or a combination of the user’s biological information (pulse, heartbeat, etc.), exercise information (steps, distance, etc.), life information (wake-up, sleep, meal, working time, break time, vacation, exercise or the like), volume data (external environmental sound, etc.) collected via the user terminal 200. As the profile information, for example, based on data of an individual user or a plurality of users, it is generated based on a model that has been machined in advance, and it is information that can be the most suitable solution for tinnitus suppression.

The electrical stimulation unit 133 electrically stimulates the user’s brain cells by applying a predetermined voltage between the first electrode 100a and the second electrode 100b having polarity. The electrical stimulation unit 133 is configured so that, for example, the current from the scalp is flowed to the resistance via the first electrode 100a, and the stimulation current from the resistor can be output to the scalp via the second electrode 100b, so that the brain cells around the second electrode 100b can be electrically stimulated. Of course, the opposite is also true.

The electrical stimulation unit 133 may appropriately change the resistance value R of the resistance based on, for example, the profile information stored in the storage unit 132. By setting the resistance value of the resistor R to, for example, 50 [Ω] to 1 [kQ], the electric stimulation unit 133 sets the current value of the stimulus current flowing between the electrodes 100a and 100b to a small amount of 1 [mA] to 5 [mA] that cannot be sensed by the user’s tactile sensation. In imparting stimulation to the brain by current, methods such as rTMS (repetitive Transcranial Magnetic Stimulation: repetitive transcranial magnetic stimulation) and tDCS (transcranial Direct Current Stimulation: transcranial DC electrical stimulation) are examples, but are not limited to this.

The output unit 140 is, for example, a speaker and a D / A converter (not shown), and for example, the sound acquired from the first input unit 110 is output to the user’s ear. The output unit 140 may output therapeutic sound for tinnitus via, for example, a pair of left and right left earphones 100L and a right earphone 100R. The therapeutic sound may include, for example, amplitude modulated sound, frequency modulated sound, utterance, music, natural sound, and the like. Natural sounds can include, for example, environmental sounds, waves, forest sounds, rain sounds, river sounds, and the like. The output unit 140 is an example of the output portion described in the billing range.

For example, the communication unit 150 transmits ambient sound data acquired from the second input unit 120 and / or audio data acquired from the first input unit 110 to the user terminal 200, and ambient sound data and / or voice sound data (hereinafter collectively referred to as “sound data”). A parameter set based on the analysis result is received from the user terminal 200, an instruction on the operation of the electrical stimulation unit 133 is accepted from the server 300, and transmitted to the storage unit 132. The communication unit 150 is an example of the instruction reception unit described in the scope of the claim. The communication unit 150 may be a near-field communication interface of Bluetooth ® and BLE (Bluetooth Low Energy), but is not limited thereto.

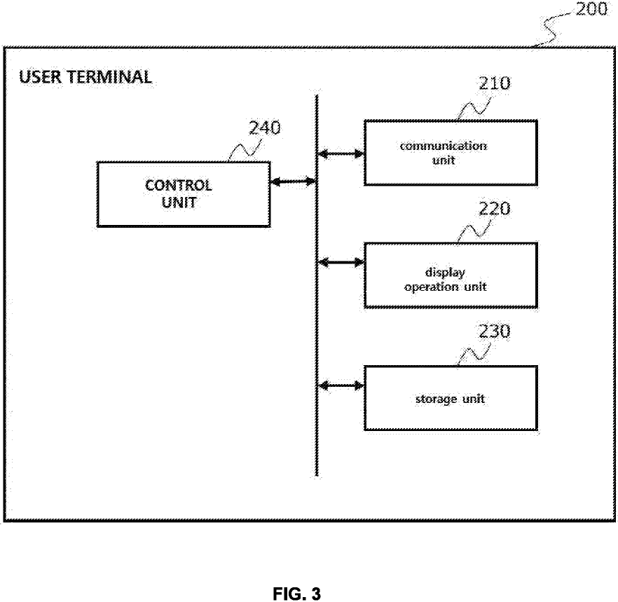

FIG. 3 is a functional block configuration diagram showing the user terminal 200 of FIG. The user terminal 200 comprises a communication unit 210, a display operation unit 220, a storage unit 230, and a control unit 240.

The communication unit 210 is a communication interface for communicating with the server 300 via the network NW, and communication is performed according to a communication agreement such as TCP / IP. When using the wireless earphone 100, the user terminal 200 is preferably in a state where communication is at least normally possible with the server 300 so that the wireless earphone 100 can be adjusted in real time.

The display operation unit 220 is a user interface used for displaying text, images, and the like according to the input information from the control unit 240, and when the user terminal 200 is configured with a tablet terminal or a smartphone, it is composed of a touch panel or the like. The display operation unit 220 is activated by a control program stored in the storage unit 230 and executed by a user terminal 200 that is a computer (electronic computer).

The storage unit 230 is a program for executing various control processes or each function in the control unit 240, input information, and the like, and is composed of RAM or ROM or the like. Further, the storage unit 230 temporarily remembers the communication contents with the server 300.

The control unit 240 controls the overall operation of the user terminal 200 by executing the program stored in the storage unit 230, and is composed of a CPU or GPU or the like.

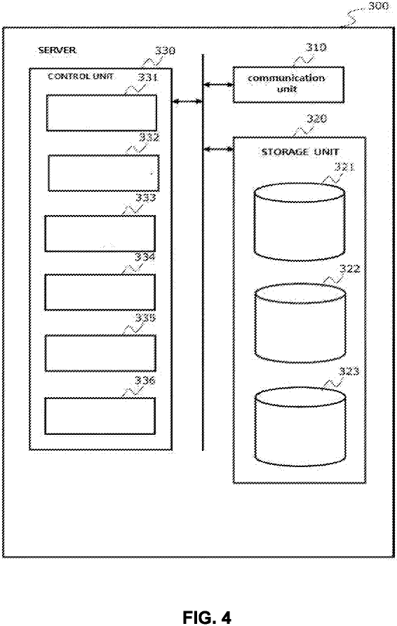

FIG. 4 is a functional block configuration diagram of the server 300 of FIG. The server 300 comprises a communication unit 310, a storage unit 320, and a control unit 330.

The communication unit 310 is a communication interface for communicating with the user terminal 200 via the network NW, and communication is performed by communication conventions such as TCP / IP (Transmission Control Protocol / Internet Protocol).

The storage unit 320 remembers a program for executing various control processes or each function in the control unit 330, input information, and the like, and is composed of RAM, ROM, or the like. Further, the storage unit 320 has a user information storage unit 321 that stores user-related information (for example, setting information of the wireless earphone 100) that is various information related to the user, a test result storage unit 322, a test result storage unit 322, an analysis result storage unit 323, and the like for storing the analysis result of sound data. Furthermore, the storage unit 320 can temporarily store information that communicates with the user terminal 200. A database (not shown) containing various information may be constructed outside the storage unit 320.

The control unit 330 controls the overall operation of the server 300 by executing the program stored in the storage unit 320, and is composed of a CPU or GPU or the like. As a function of the control unit 330, the instruction reception unit 331 that accepts instructions from the user, the user information management unit 332 that refers to and processes user-related information that is various information related to the user, the confirmation test management unit 333, which performs a predetermined confirmation test or refers to the test result, and analyzes the test results of the confirmation test, It has a parameter set generation unit 334 for generating a parameter set, a sound data analysis unit 335 for analyzing input sound data, referencing and processing analysis results, and having an analysis result management unit 336, and the like. The instruction reception unit 331, the user information management unit 332, the confirmation test management unit 333, the parameter set generation unit 334, the sound data analysis unit 335, and the analysis result management unit 336 are activated by a program stored in the storage unit 320 and executed by a server 300 that is a computer (electronic computer).

The instruction reception unit 331 accepts the instruction when the user makes a predetermined request via a user interface such as an application software screen or a web screen displayed in the user terminal 200 or via various sensors provided in the wireless earphone 100.

The user information management unit 332 manages user-related information and performs predetermined processing as necessary. The user-related information is, for example, user ID, e-mail address information, and the user ID may be associated with the analysis result of sound data as a result of the confirmation test, and may be able to be confirmed on the application.

The confirmation test management unit 333 executes a predetermined confirmation test (described later in the flowchart), refers to the results of the confirmation test, and executes a predetermined process (for example, displaying the confirmation test result on the user terminal 200, transmitting the result to the parameter set generation unit 334, etc.).

The parameter set generator 334 generates a setting value that increases or decreases the gain (amplification amount) for a plurality of predetermined frequencies (eg, 8 or 16 channels) based on the results of the above-described confirmation test and / or the analysis results of the sound data described later.

The sound data analysis unit 335 analyzes the input sound data. Here, the analysis of the sound data is, for example, to analyze the sound data input using the Fast Fourier Transform and to determine that the noise of a specific frequency (eg, frequency from location such as on a train, airplane, city, frequency derived from a human voice, a source such as a television) has come out stronger than a predetermined reference value. When determined, the determination result may be transmitted to the parameter set generation unit 334. In addition, noise of a specific frequency may be stored by corresponding to each as a hearing mode, and further, it may be configured to manually set the hearing mode by the user.

The analysis result management unit 336 refers to the analysis result of the sound data, performs a predetermined process (for example, displaying the analysis result on the user terminal 200, transmitting the result to the parameter set generation unit 334, and the like).

### Flow of < Processing>

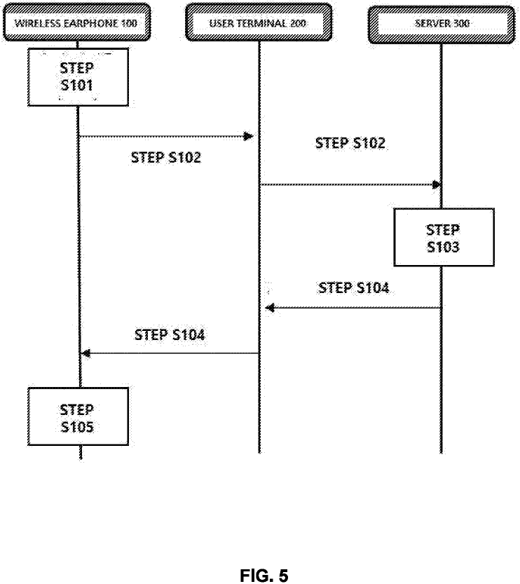

Referring to FIG. 5, the process flow for adjusting the wireless earphones executed by the system of the first embodiment of the present disclosure will be described. FIG. 5 is an example of a flowchart according to the method of adjusting the wireless earphone according to the first embodiment of the present disclosure. In addition, although the flowchart performs a test for initial setting, the test may be performed at any timing as well as the initial setting, or the test may not be performed depending on the user.

First, before using the wireless earphone 100, a test for initial setting is performed (step S101). For example, on an application launched on the user terminal 200, a confirmation test for hearing at multiple predetermined frequencies (for example, 16 channels) (for example, if you hear a “pea” sound for each frequency, a test to press the OK button) is performed, and a parameter set is generated based on the test result. The gain (amplification amount) for each frequency is stored in the user terminal 200 as a parameter set, and based on it, for example, the gain (amplification amount) for each frequency of the wireless earphone is set by DSP.

Next, the wireless earphone 100 acquires sound data from the first input unit 110 and / or the second input unit 120 and transmits it to the server 300 via the user terminal 200 (step S102).

Next, the server 300 performs analysis of sound data by the sound data analysis unit 335 and generates a parameter set (step S103).

Next, the server 300 transmits a parameter set to the wireless earphone 100 via the user terminal 200, stores it in the storage unit 132, and further adjusts the gain (amplification amount) for each frequency of the wireless earphone by, for example, DSP (step S105). Steps S102-105 are performed every predetermined sample time.

Thereby, by adjusting in real time according to the sound input to the wireless earphone (especially the sound of the surrounding environment), it is possible to finely adjust the input sound and output it to the user, and the user is always easy to hear.

Next, an example of a function for reducing tinnitus will be described while referring to FIG.

First, the application is started on the user terminal 200 and the tinnitus reduction function is started (S201).

Next, the electrical stimulation unit 133 applys a predetermined voltage between the first electrode 100a and the second electrode 100b based on the operation instruction received by the communication unit 150 from the server 300 (S202). One of the first electrode 100a and the second electrode 100b is a positive electrode (anode), and the other electrode can be a negative electrode (cathode). The electrical stimulation unit 133 reads out the profile information related to the user’s tinnitus from the storage unit 132 and estimates the user’s physical condition or mental state based on the profile information. Depending on the results of the estimation, the electrical stimulation unit 133 may reduce or strengthen the stimulation current within the range of 1 [mA] to 5 [mA]. For example, when the user is doing well, the electrical stimulation unit 133 can increase the stimulation current. For example, if the user is unwell, the electrical stimulation unit 133 reduces the stimulation current, and the output unit 140 may output sound or music that can perform healing effects such as the sound of flowing rivers and waves via the left earphone 100L and the right earphone 100R. Of course, either the energization of a weak stimulation current or the sound output that can perform a healing effect may be either, and it can be set according to the user’s physical condition or mental state.

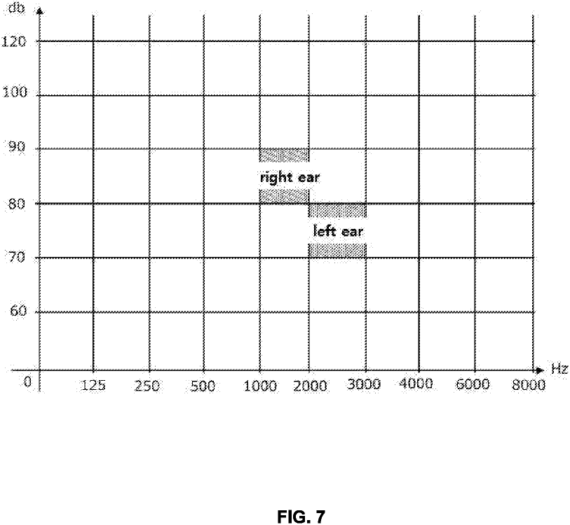

Next, a sound similar to tinnitus is selected from the table of intensity (db) and frequency (Hz) as shown in FIG. 7 presented in the application (S203). As shown in FIG. 7, it is possible to listen to the sound of the selection position by selecting in the table. In addition, especially if there are symptoms of tinnitus in both the right ear and the left ear, select them respectively.

Next, the sound of the reverse phase of the selected sound is set as a therapeutic sound (S204).

Thereafter, for example, by outputting a therapeutic sound from the wireless earphone 100, it is possible to cancel the tinnitus sound and reduce tinnitus. In particular, it is even more useful for users with different tinnitus intensity or frequency on the left and right. For example, it is possible to select a sound of 2000 Hz and 70 db in the left ear, select a sound of 1000 Hz and 80 db in the right ear, and set the sound of the opposite phase as a therapeutic sound. Here, as a therapeutic sound, a sound or music that can play a healing effect such as the sound flowing of the river and the sound of waves may be output via the left earphone 100L and the right earphone 100R.

Since conventional tinnitus treatment methods attempt to reduce tinnitus by pronunciation of therapeutic sounds, strict hearing is required. For this reason, for example, it is not suitable when used by a person who is damaging auditory cells.

In this regard, according to the transcranial current stimulation system 1 according to the present disclosure, a predetermined voltage can be applied through two electrodes 100a and 100b that can be attached to the user’s scalp in place, so that no matter what the auditory cell is, it is possible to reduce tinnitus by noninvasively applying electrical stimulation to brain cells. In addition, not only tinnitus reduction, but also treatment of schizophrenia or brain training for dementia patients can be expected.

Here, in this system 1, first, the wireless earphone outputs a therapeutic sound, and the server 300 can accept feedback from the user terminal 200 (such as “OK” or “NG” ) whether tinnitus is reduced, and the user feedback result is determined to be insufficient, as described in S202, as described in S202, Instructions for applying a voltage can be sent to the wireless earphone. Thereby, tinnitus reduction can be realized in a noninvasive manner without giving unnecessary load to the user.

Here, in the system 1, the server 300 can receive feedback from the user terminal 200 every certain period (for example, every day, 3 days, and every week). Based on the feedback, the server 300 may adjust the therapeutic sound output to the wireless earphone, change the sound or music selected as the treatment sound, or adjust the amount of current given to the user.

The above, embodiments pertaining to disclosure have been described, but these can be implemented in various other forms, and various omissions, substitutions, and modifications can be performed. These embodiments and variants and those that have been omitted, replaced and modified are included in the technical scope of the claims and their even scope.

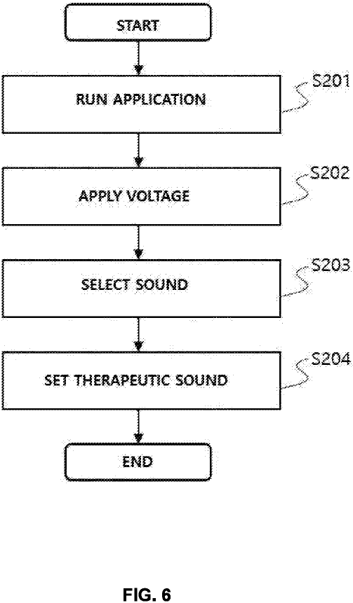

In this system 1, after applying a series of tinnitus reduction methods by sound and/ or current stimulation as shown in FIG. 6, feedback information on tinnitus may be acquired from the user terminal 200 by, for example, a method such as a survey, every certain period (eg, every day, 3 days, every week).

Based on the feedback information, the server 300 may improve the treatment method by sound and / or current stimulation, may change the frequency of the sound selected as the treatment sound, music, natural sound, or the like.

A series of processing with the apparatus described herein may be realized using software, hardware, and any combination of software and hardware. A computer program for realizing each function of the user terminal 200 according to the present embodiment can be prepared and implemented on a PC or the like. It can also provide a computer-readable recording medium in which such a computer program is stored. The recording medium is, for example, a magnetic disk, an optical disk, an optical magnetic disk, a flash memory, and the like. Further, the above-described computer program may be delivered via a network, for example, without using a recording medium.

Further, the process described using the flowchart diagram herein may not necessarily be performed in the order shown. Some processing steps may be executed in parallel. Additional processing steps may be employed, and some processing steps may be omitted.

In addition, the effects described herein are illustrative or exemplific, and are not limited. That is, the techniques pertaining to the present disclosure may have other effects that are clear to those skilled in the art from the description herein, along with or in lieu of the above effects.

### REFERENCE SIGNS LIST

1 Transcranial current stimulation system
100 Wireless Earphones
100a First electrode
100b Second electrode
100L Left Earbuds
100R Right Earphone
132 Memory
140 Output
150 Communication Department (Instruction Reception)
200 user terminals
300 server equipment
NW Network

1. Wireless earphones having a pair of left and right left earphones and right earphones that are directly or indirectly connected to the server over the network and acquire and output sound data from the server, wireless earphones comprising:
a first electrode that is provided in a predetermined position of the left earphone and can come into contact with the left head of the user wearing the left earphone;
a second electrode that is provided in a predetermined position of the right earphone and can come into contact with the right head of the user wearing the right earphone;
a predetermined voltage is applied between the first electrode and the second electrode, and an electrical stimulation unit that electrically stimulates the user’s brain cells is provided.

2. The wireless earphones of claim 1, further comprising:
an instruction reception unit that accepts instructions for the operation of the electrical stimulation unit from the server,
wherein the electrical stimulation unit applys a predetermined voltage between the first electrode and the second electrode based on the instruction received by the instruction reception unit.

3. The wireless earphones of claims 1-2, further comprising:
an output unit that outputs a treatment sound for tinnitus via the left and right pair of left and right earphones and the right earphone.

4. The wireless earphones of claim 1, further comprising:
an equipped with a storage unit that remembers profile information about the user’s tinnitus,
wherein based on the profile information, the electrical stimulation unit applys a predetermined voltage between the first electrode and the second electrode.

5. The wireless earphones of claim 1, wherein the stimulation current flowing between the user’s scalp and the first electrode and the second electrode is 1 to 5 mA.
